(19) United States
(12) Patent Application Publication
(10) Pub. No.: US 2025/0243344 A1
(43) Pub. Date: Jul. 31, 2025

(54) COMPOUND, RUBBER BLEND CONTAINING THE COMPOUND, VEHICLE TIRE COMPRISING THE RUBBER BLEND IN AT LEAST ONE COMPONENT, PROCESS FOR PREPARING THE COMPOUND, AND USE OF THE COMPOUND AS AN ANTI-AGING AGENT AND/OR ANTIOXIDANT

(71) Applicant: Continental Reifen Deutschland GmbH, Hannover (DE)

(72) Inventors: Andreas Jacob, Hannover (DE); David-Raphael Dauer, Dassel (DE); Julian Strohmeier, Einbeck (DE); Anna-Lena Dreier, Hannover (DE); Frank Fleck, Wennigsen (DE); Carla Recker, Hannover (DE)

(73) Assignee: Continental Reifen Deutschland GmbH, Hannover (DE)

(21) Appl. No.: 18/856,052

(22) PCT Filed: Mar. 20, 2023
(86) PCT No.: PCT/DE2023/200062

§ 371 Date: Oct. 10, 2024

(30) Foreign Application Priority Data
Apr. 13, 2022 (DE) 10 2022 203 732.4

(51) Int. Cl. C08K 5/3417; B60C 1/00; C07D 209/88; C08L 7/00

(52) U.S. Cl. CPC C08K 5/3417; B60C 1/0016; B60C 1/0025; C07D 209/88; C08L 7/00; C08L 2201/08

(57) ABSTRACT

A compound, a rubber mixture containing the compound, a vehicle tire comprising the rubber mixture in at least one component, a process for producing the compound and the use of the compound as an aging stabilizer and/or antioxidant. The compound has the formula (I)

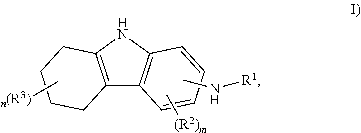

wherein R1 is selected from the group consisting of xi) aromatic radicals, wherein the aromatic radicals optionally bear substituents selected from the group consisting of halogen radicals, cyano radicals, ester radicals, ketone radicals, ether radicals and thioether radicals, and xii) linear, branched and cyclic aliphatic C3—to C12—radicals.

### DESCRIPTION

The invention relates to a compound, to a rubber mixture containing the compound, to a vehicle tire comprising the rubber mixture in at least one component, to a process for producing the compound and to the use of the compound as an aging stabilizer and/or antioxidant.

It is known that polymeric materials such as, in particular, rubbers are used in vehicle tires and industrial rubber articles.

Natural rubber and synthetic polymers (such as IR, BR, SSBR, ESBR etc.) but also natural and synthetic oils, fats and lubricants are subject to oxidation reactions which have an adverse effect on the originally desired properties upon prolonged storage and in particular in the target application which is often at elevated temperatures. Depending on the type of the polymer, the polymer chains are shortened right up to liquefaction of the material, or the material subsequently hardens.

Aging stabilizers thus contribute to a crucial degree to the service life of vehicle tires and other industrial rubber articles.

Known aging stabilizers include aromatic amines, for example
6PPD (N-(1,3-dimethylbutyl)-N′-phenyl-p-phenylenediamine),
IPPD (N-isopropyl-N′-phenyl-p-phenylenediamine) or
SPPD (N-(1-phenylethyl)-N′-phenyl-p-phenylenediamine).

These molecules can react with oxygen or ozone or free radicals formed, such as alkyl, alkoxy and alkylperoxy radicals, and thus scavenge these and accordingly protect the polymers from further oxidation reactions.

However, a disadvantage of this substance class is that they could be carcinogenic.

Aging stabilizers which especially react with ozone and effect scavenging thereof are also referred to as “antiozonants”.

A further problem in relation to aging stabilizers is that of undesired blooming. Here, the somewhat poor solubility of the molecules of the aging stabilizer in the surrounding polymer matrix of the rubber article causes them to diffuse to the surface of the article to be protected and form a film there that is usually distinguishable in color from the rest of the article. In the case of vehicle tires, this usually manifests itself in a brown coloring of the otherwise black sidewall. In addition to the aesthetic disadvantages this is also associated with disadvantages in terms of the aging stabilization effect. This is because bloomed substances are usually removed. This firstly reduces the total amount of aging stabilizer and also has the effect that further molecules of the aging stabilizer diffuse into their place, with the result that the polymers have an ever falling level of protection.

It is an object of the invention to provide a novel compound which can especially be used as an aging stabilizer in vehicle tires or other industrial rubber articles, specifically with a lower hazard potential coupled with sufficient solubility in the respective matrix, for example and in particular in the polymer. This is intended to ensure continuing optimal protection from oxygen and ozone and to prevent the tendency for blooming while reducing hazardousness to health.

The compound is at the same time intended to ensure comparable or even improved aging stabilization compared to aromatic amines, such as 6PPD.

The object is achieved by the compound according to the invention as claimed in claim 1, by the rubber mixture according to the invention containing the compound, and by the vehicle tire according to the invention comprising the rubber mixture according to the invention in at least one component.

The object is also achieved by the process according to the invention for producing the compound according to the invention.

The compound as claimed in claim 1 has the general formula I):

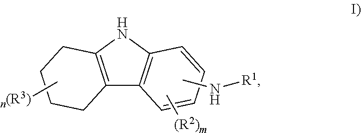

wherein R¹ is selected from the group consisting of xi) aromatic radicals, wherein the aromatic radicals optionally bear substituents selected from the group consisting of halogen radicals, cyano radicals, ester radicals, ketone radicals, ether radicals and thioether radicals,
and xii) linear, branched and cyclic aliphatic C₃-to C₁₂-radicals, wherein the radicals R² and R³ may independently of one another be identical or different and are selected from the group consisting of linear, branched and cyclic, saturated and unsaturated, aliphatic C₁-to C₁₂-radicals optionally bearing one or more halogen substituents, aryl radicals optionally bearing one or more halogen substituents, and halogen radicals, wherein fluorine, bromine and chlorine are preferred, cyano radicals, ester radicals, ketone radicals, ether radicals and thioether radicals
and wherein m assumes the value 0 or 1 or 2 or 3
and wherein n assumes the value 0 or is an integer from 1 to 8, preferably 1 to 4.

It is apparent to those skilled in the art that, for example, when m is 0 (zero) or 1 or 2 a respective hydrogen atom is bonded to the corresponding carbon atom of the benzene ring instead of R^{2.} This of course applies analogously for R³ and n less than 8.

It will likewise be apparent to those skilled in the art that the representation of the bonds of (R²)ₘ and (R³)ₙ and R¹HN to the respective ring of the structure means that these groups may each be disposed at any position on the respective ring whilst observing the tetravalency of the carbon atom.

In the context of the present invention descriptions of the kind “C₃-to C₁₂-radicals” are to be understood as meaning radicals having 3 to 12 carbon atoms. Irrespective of this “C₁” is used to describe the position of the most highly oxidized carbon atom/the highest priority according to the Cahn-Ingold-Prelog convention (CIP). It will be clear to a person skilled in the art what is meant in the respective context.

The compound according to the invention is a tetrahydrocarbazole derivative and exhibits a lower hazard potential relative to known aging stabilizers based on aniline (possible cleavage product of 6PPD).

This is a crucial advantage, especially in a technical application such as in vehicle tires or other industrial rubber products, since the rubber ingredients can be liberated by abrasion or other degradation processes.

Compared to the known aging stabilizer 6PPD the compound according to the invention exhibits a comparable or even improved antioxidant effect, as a result of which the compound of formula I) achieves a comparable or even improved protective effect, in particular in vehicle tires and other industrial rubber articles, but also in oils and lubricants.

However, the invention shall not be bound to a particular mechanism of action or a particular explanation.

The compound according to the invention is thus suitable as a substitute for 6PPD, the degradation products of which are extremely toxic to silver salmon and thus probably also to other aquatic organisms.

The compound of the invention additionally has very good solubility in rubber mixtures, especially for vehicle tires and other industrial rubber articles. This avoids a blooming of this compound such as is familiar from many aging stabilizers which in turn means an advantageous/improved aging stabilization effect. The less an aging stabilizer blooms, the less aging stabilizer is intentionally or unintentionally removed from the surface of the article to be protected and the less aging stabilizer in turn diffuses to the surface.

The inventive compound of formula I) is particularly suitable as an aging stabilizer and/or antiozonant in vehicle tires and/or other industrial rubber articles, such as especially an air spring, a bellows, a conveyor belt, a strap, a drive belt, a hose, a rubber band, a profile, a seal, a membrane, tactile sensors for medical applications or robotics applications or a shoe sole or parts thereof and/or oils and/or lubricants.

The compound of formula I) according to the invention is particularly suitable for producing a rubber article, in particular an air spring, a bellows, a conveyor belt, a strap, a drive belt, a hose, a rubber band, a profile, a seal, a membrane, tactile sensors for medical applications or robotics applications or a shoe sole or parts thereof.

For use of the compound of formula I) in the recited articles or substances, said compound is used in a composition and used incorporated in said composition.

In vehicle tires or other industrial rubber articles, said composition is in particular a rubber mixture.

The invention further provides for the use of the compound of formula I) according to the invention in oils and lubricants, such as in particular fuels or fluid engines. In particular, the compound according to the invention may be used in engines.

The invention encompasses all of the advantageous embodiments reflected inter alia in the claims. The invention especially also comprises embodiments which result from a combination of different features with different levels of preference for these features so that the invention also comprises a combination of a first feature described as “preferred” or described in the context of an advantageous embodiment with a further feature described for example as “particularly preferred”.

In addition, all information relating to the features of the inventive compound also applies to the inventive process for producing the compound and the inventive rubber mixture containing the compound as well as the inventive uses.

The −N(H)R¹ group is preferably arranged in the para position relative to N(H).

The compound according to the invention thus preferably has the structure of formula II):

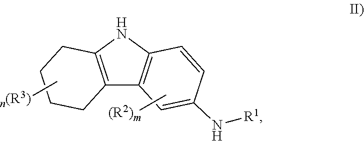

wherein R¹, R², R³ and m and n are as defined above.

This achieves the object of the invention particularly well and especially achieves a very good solubility of the compound in rubber mixtures, in particular for vehicle tires.

The radicals R² and R³ are independently of one another identical or different and are selected from the group consisting of linear, branched and cyclic, saturated and unsaturated, aliphatic C₁-to C₁₂-radicals optionally bearing one or more halogen substituents, aryl radicals optionally bearing one or more halogen substituents, and halogen radicals, wherein fluorine, bromine and chlorine are preferred, cyano radicals, ester radicals, ketone radicals, ether radicals and thioether radicals.

The recited radicals R² and R³ may in particular already be bonded to the respective ring/the precursor thereof through selection of suitable starting substances.

The tetravalency of carbon means that m can assume a maximum value of 3. Accordingly, up to three substituents R² may be bonded to the benzene ring.

The tetravalency of carbon means that n can assume a maximum value of 8. Accordingly, up to eight substituents R³ may be bonded to the cyclohexene ring.

However, in preferred embodiments n is an integer from 1 to 4, wherein each carbon atom preferably has only one radical R³ bonded to it.

This makes the molecule more stable due to lower steric crowding. The compound where n is 0 or 1 to 4 is moreover easier to synthesize since a synthetically easily obtainable precursor comprises a benzene ring, which can accordingly only introduce up to four radicals R^{3,} instead of the cyclohexyl ring.

It is preferable when, for example and especially in formulae I) and II), m is zero (0).

It is preferable when, for example and especially in formulae I) and II), n is zero (0).

This results in the preferred structures shown in formulae Ia) or IIa):

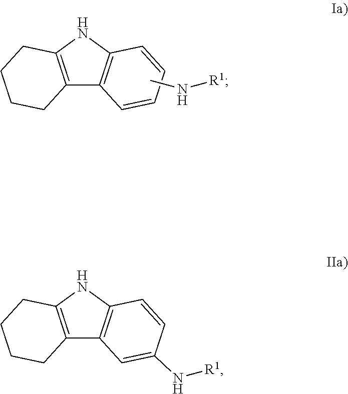

wherein R¹ is as defined above.

The radical R¹ is selected from the group consisting of
xi) aromatic radicals, wherein the aromatic radicals optionally bear substituents selected from the group consisting of halogen radicals, cyano radicals, ester radicals, ketone radicals, ether radicals and thioether radicals,
and xii) linear, branched and cyclic aliphatic C₃-to C₁₂-radicals.

The aromatic radical of subgroup xi) is by way of example and preferably selected from phenyl radicals (−C₆H₅) and benzyl radicals (−CH₂−C₆H₅), wherein phenyl is particularly preferred.

The aromatic radicals of subgroup xi) may bear substituents.

As mentioned above these are selected from the group consisting of halogen radicals, cyano radicals, ester radicals, ketone radicals, ether radicals and thioether radicals.

It is preferable when the substituents are selected from the group consisting of ester radicals, ketone radicals, ether radicals and thioether radicals.

In preferred embodiments the aromatic radical is not substituted at the two carbon atoms adjacent to the C₁ atom, i.e. the carbon atom bonded to the N atom. In the case of a benzene ring as the basic structure it is thus preferable when there is no substituent in the ortho-position to the N atom.

In further preferred embodiments the aromatic radical of subgroup xi) is not substituted.

It is preferable when R¹ is bonded to the nitrogen atom (N) via a tertiary carbon atom. This means that the C₁ atom is preferably a tertiary carbon atom.

In the context of the present invention the term “tertiary carbon atom” is to be understood as meaning a carbon atom which is bonded to only one hydrogen atom.

This results—relative to secondary and quaternary carbon atoms—in a particularly good protective effect due to the presence of the compound in rubber mixtures, in particular for vehicle tires and other technical rubber articles, and in particular in optimal reactivity in connection with the mechanisms relevant for aging stabilization, wherein undesired side reactions are avoided.

In further advantageous embodiments, especially in the aforementioned formulae I), II), Ia), IIa), R¹ is a branched or cyclic alkyl radical having three to twelve carbon atoms, preferably three to eight carbon atoms, wherein R¹ is particularly preferably selected from 1,3-dimethylbutyl and cyclohexyl radicals, wherein R¹ is very particularly preferably a 1,3-dimethylbutyl radical.

This achieves a particularly good solubility in rubber mixtures for vehicle tires and other industrial rubber articles.

In a particularly preferred embodiment the compound according to the invention has the structure of formula III):

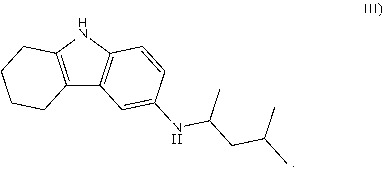 [SMILES: CC(C)CC(C)Nc1ccc2[nH]c3c(c2c1)CCCC3]

The compound of formula III) has a particularly good solubility in polymers, especially in rubber mixtures for vehicle tires and other industrial rubber articles. At the same time the compound of formula II) may be produced in a particularly simple, energy-efficient and cost-saving manner and, compared to 6PPD, exhibits a comparable or even improved antioxidant effect and thus a comparable or improved aging stabilization effect.

According to IPUAC nomenclature the compound of formula III) may also be referred to as
6-(1,3-dimethylbutylamino)-2,3,4,9-tetrahydro-1H-carbazole.

As mentioned above the present invention further provides a process for producing the compound of formula I) which comprises at least the following process steps:
i) producing or providing the substance of formula B1):

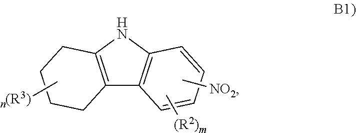

ii) reacting the compound of formula B1) with hydrogen and a ketone or aldehyde, preferably ketone, to afford the compound of formula I):

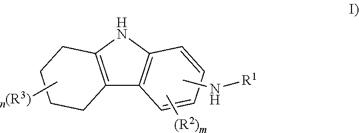

wherein R¹ is selected from the group consisting of xi) aromatic radicals, wherein the aromatic radicals optionally bear substituents selected from the group consisting of halogen radicals, cyano radicals, ester radicals, ketone radicals, ether radicals and thioether radicals,
and xii) linear, branched and cyclic aliphatic C₃-to C₁₂-radicals,
wherein the radicals R² and R³ may independently of one another be identical or different and are selected from the group consisting of linear, branched and cyclic, saturated and unsaturated, aliphatic C₁-to C₁₂-radicals optionally bearing one or more halogen substituents, aryl radicals optionally bearing one or more halogen substituents, and halogen radicals, wherein fluorine, bromine and chlorine are preferred, cyano radicals, ester radicals, ketone radicals, ether radicals and thioether radicals
and wherein m assumes the value 0 or 1 or 2 or 3
and wherein n assumes the value 0 or is an integer from 1 to 8, preferably 1 to 4.

All of the above information provided in connection with the description of the compound according to the invention, including preferred embodiments, and having regard to any possible hierarchy of preference for these features and the combination of these features, applies to R¹, R², R³, m and n.

The compound of formula B1) is producible for example analogously to B.A. Dalvi, Tet. Lett., 2018, 59, 2145-2149 according to the following scheme S1):

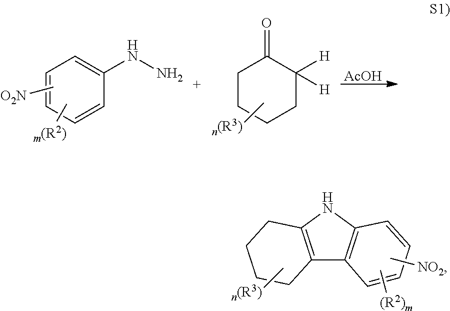

wherein AcOH stands for acetic acid.

All the above information applies to R¹, R², R³, m and n, wherein in the cyclohexanone derivative one of the carbon atoms adjacent to the carbonyl group bears no radical R³. In scheme S1) this carbon atom is characterized in that the two hydrogen atoms are explicitly shown as bonded.

The group −N(H)R¹ in formula I) and the group −NO₂ in the starting substance B1) are preferably arranged in the para position relative to N(H) and N(H)—N(H)₂ respectively as specified above.

The compound produced according to the invention thus preferably has the structure of formula II):

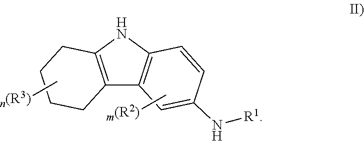

To this end the starting substance of formula B1) preferably has the structure of formula B1a):

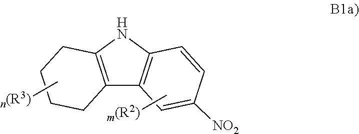

wherein R¹, R², R³ and m and n are defined as above, including all embodiments and combinations of features.

It is preferable when the reaction with hydrogen in step ii) employs a suitable catalyst, referred to in the context of the present invention as “hydrogenation catalyst”.

It is preferable when the hydrogenation catalyst is a noble metal catalyst, such as in particular palladium (Pd) or platinum (Pt). It is preferable when the noble metal is used on carbon (C), such as palladium on carbon (Pd/C).

It is further also possible to use other known catalysts, such as Raney nickel or copper chromite.

It is particularly preferable when the reaction in step ii) is therefore carried out with hydrogen
using a hydrogenation catalyst.

The reaction in step ii) is preferably carried out at a temperature of 50° C. to 130° C., preferably 50° C. to 100° C., particularly preferably 50° C. to 80° C., especially 60° C. for example.

It is preferable when the reaction mixture is subjected to hydrogen at a pressure of 10 to 70 bar, particularly preferably 10 to 30 bar, in particular 20 bar for example, and then preferably stirred for 1 to 20 hours, preferably 3 to 13 hours, particularly preferably 5 to 13 hours, in particular 10 hours for example.

The reaction with hydrogen in step ii) is preferably carried out in a vessel suitable for the preferably relatively high pressure, such as especially in an autoclave or in another pressure reactor.

It is particularly preferable when the reaction in step ii) is carried out with hydrogen using a hydrogenation catalyst and at a temperature of 50° C. to 130° C., preferably 50° C. to 100° C., particularly preferably 50° C. to 80° C., and wherein the reaction mixture is subjected to hydrogen at a pressure of 10 to 70 bar, particularly preferably 10 to 30 bar, in particular 20 bar for example, and the reaction is carried out in an autoclave or in another pressure reactor.

The ketone in step ii) is the ketone derivative of the subsequent radical R¹; in the case of an aldehyde it is accordingly the aldehyde derivative.

For the sake of simplicity, the abbreviated formula R¹=O is used for the aldehyde or ketone since the radical R¹ is the part that remains on the nitrogen atom after the reaction with the aldehyde or ketone.

The ketone used here is preferably methyl isobutyl ketone.

The solvent in step ii) may be either the ketone or aldehyde, if this is in liquid form, or an inert solvent, such as toluene or xylene, especially if the ketone or aldehyde is in solid form. In the latter case the ketone or aldehyde is employed as reactant only in stoichiometric amounts.

It is preferable to use a ketone or aldehyde R¹=O, particularly preferably ketone, in liquid form as solvent. This makes it possible to dispense with any additional substance, such as toluene or xylene.

Step ii) is preferably followed by a purification, for example by filtration and scrubbing with a solvent, in particular ethanol, and/or by column chromatography, for example on silica gel or by recrystallization from cyclohexane or longer-chain aliphatics.

As mentioned above, the invention further provides a rubber mixture.

The rubber mixture according to the invention contains the compound of formula I), for example and preferably the compound of formula III). The rubber mixture according to the invention may in principle be any rubber mixture in which in particular the novel compound of formula I) or III) according to the invention acts as an aging stabilizer and/or antiozonant with low toxicity.

The rubber mixture according to the invention contains at least one rubber.

It is preferable when the rubber mixture according to the invention contains 0.1 to 10 phr, particularly preferably 0.1 to 7 phr, very particularly preferably 1 to 6 phr, in turn preferably 1 to 3 phr, of the compound of formula I), for example and preferably the compound of formula III).

The rubber mixture may also contain a mixture of two or more compounds conforming to formula I).

The unit “phr” (parts per hundred parts of rubber by weight) used in this document is the conventional indication of quantity for mixture recipes in the rubber industry. The dosage of the parts by weight of the individual substances is based in this document on 100 parts by weight of the total mass of all high molecular weight (M_{w} greater than 20 000 g/mol) rubbers present in the mixture.

In advantageous embodiments of the invention, the rubber mixture of the invention contains at least one diene rubber.

The rubber mixture may accordingly contain a diene rubber or a mixture of two or more different diene rubbers.

Diene rubbers are rubbers which are formed by polymerization or copolymerization of dienes and/or cycloalkenes and thus have C=C double bonds either in the main chain or in the side groups.

The diene rubber is preferably selected from the group consisting of natural polyisoprene (NR), synthetic polyisoprene (IR), epoxidized polyisoprene (ENR), butadiene rubber (BR), butadiene-isoprene rubber, solution-polymerized styrene-butadiene rubber (SSBR), emulsion-polymerized styrene-butadiene rubber (ESBR), styrene-isoprene rubber, liquid rubbers having a molecular weight M_{w} of more than 20 000 g/mol, halobutyl rubber, polynorbornene, isoprene-isobutylene copolymer, ethylene-propylene-diene rubber, nitrile rubber, chloroprene rubber, acrylate rubber, fluoro rubber, silicone rubber, polysulfide rubber, epichlorohydrin rubber, styrene-isoprene-butadiene terpolymer, hydrogenated acrylonitrile butadiene rubber and hydrogenated styrene-butadiene rubber.

Nitrile rubber, hydrogenated acrylonitrile-butadiene rubber, chloroprene rubber, butyl rubber, halobutyl rubber and/or ethylene-propylene-diene rubber in particular are used in the production of industrial rubber articles, such as straps, drive belts and hoses, and/or shoe soles. The mixture compositions known to those skilled in the art for these rubbers, which are specific in terms of fillers, plasticizers, vulcanization systems and additives, are preferably used here.

The natural and/or synthetic polyisoprene of all embodiments may be either cis-1,4-polyisoprene or 3,4-polyisoprene. However, the use of cis-1,4-polyisoprenes having a cis-1,4 proportion of >90% by weight is preferred. Such a polyisoprene is firstly obtainable by stereospecific polymerization in solution with Ziegler-Natta catalysts or using finely divided lithium alkyls. Secondly, natural rubber (NR) is one such cis-1,4-polyisoprene, for which the cis-1,4 content in the natural rubber is greater than 99% by weight.

A mixture of one or more natural polyisoprenes with one or more synthetic polyisoprenes is further also conceivable.

In the context of the present invention the term “natural rubber” is to be understood as meaning naturally occurring rubber which may be obtained from Hevea rubber trees and from “non-Hevea” sources. Non-Hevea sources include for example guayule shrubs and dandelion such as for example TKS (Taraxacum kok-saghyz; Russian dandelion).

If the rubber mixture of the invention contains butadiene rubber (i.e. BR, polybutadiene), this may be any of the types known to those skilled in the art. These include what are called the high-cis and low-cis types, with polybutadiene having a cis content of not less than 90% by weight being referred to as the high-cis type and polybutadiene having a cis content of less than 90% by weight being referred to as the low-cis type. An example of a low-cis polybutadiene is Li-BR (lithium-catalyzed butadiene rubber) having a cis content of 20% to 50% by weight. Particularly good properties and low hysteresis of the rubber mixture are achieved with a high-cis BR.

The polybutadiene(s) used may be end group-modified with modifications and functionalizations and/or be functionalized along the polymer chains. The modification may be selected from modifications with hydroxyl groups and/or ethoxy groups and/or epoxy groups and/or siloxane groups and/or amino groups and/or aminosiloxane and/or carboxyl groups and/or phthalocyanine groups and/or silane-sulfide groups. However, further modifications known to those skilled in the art, also referred to as functionalizations, are also useful. Metal atoms may be a constituent of such functionalizations.

In the case where at least one styrene-butadiene rubber (styrene-butadiene copolymer) is present in the rubber mixture this may be solution-polymerized styrene-butadiene rubber (SSBR) and emulsion-polymerized styrene-butadiene rubber (ESBR), a mixture of at least one SSBR and at least one ESBR also being employable. The terms “styrene-butadiene rubber” and “styrene-butadiene copolymer” are used synonymously in the context of the present invention.

The styrene-butadiene copolymer used may be end group-modified and/or functionalized along the polymer chains with the modifications and functionalizations recited above for the polybutadiene.

The at least one diene rubber is preferably selected from the group consisting of natural polyisoprene (NR, natural rubber), synthetic polyisoprene (IR), butadiene rubber (BR), solution-polymerized styrene-butadiene rubber (SSBR), emulsion-polymerized styrene-butadiene rubber (ESBR), butyl rubber (IIR) and halobutyl rubber.

In a particularly preferred embodiment of the invention, the at least one diene rubber is selected from the group consisting of natural polyisoprene (NR), synthetic polyisoprene (IR), butadiene rubber (BR), solution-polymerized styrene-butadiene rubber (SSBR) and emulsion-polymerized styrene-butadiene rubber (ESBR).

In a particularly advantageous embodiment of the invention, the rubber mixture comprises at least one natural polyisoprene (NR), preferably in amounts of 50 to 100 phr, and in one particularly advantageous embodiment of the invention 80 to 100 phr, even more preferably 95 to 100 phr, preferably in turn 100 phr. Such a rubber mixture especially shows optimized tear properties and abrasion properties coupled with good processibility and reversion stability.

If the rubber mixture contains less than 100 phr of NR, it preferably contains, as a further rubber, at least one diene rubber selected from the group consisting of synthetic polyisoprene (IR), butadiene rubber (BR), solution-polymerized styrene-butadiene rubber (SSBR) and emulsion-polymerized styrene-butadiene rubber (ESBR).

In a further particularly advantageous embodiment of the invention, the rubber mixture comprises at least one natural polyisoprene (NR), preferably in amounts of 5 to 55 phr, and in one particularly advantageous embodiment of the invention 5 to 25 phr, even more preferably 5 to 20 phr. Such a rubber mixture especially exhibits good processibility and reversion stability and optimized tear properties and optimal rolling resistance characteristics.

In a further particularly advantageous embodiment of the invention, the rubber mixture comprises at least one polybutadiene (BR, butadiene rubber), preferably in amounts of 10 to 80 phr, more preferably 10 to 50 phr, and in a particularly advantageous embodiment of the invention 15 to 40 phr. This achieves particularly good tear and abrasion properties of the rubber mixture of the invention and optimal braking characteristics.

In a further particularly advantageous embodiment of the invention, the rubber mixture comprises at least one solution-polymerized styrene-butadiene rubber (SSBR), preferably in amounts of 10 to 80 phr, more preferably 30 to 80 phr, and in one particularly advantageous embodiment of the invention 50 to 70 phr. This achieves particularly good rolling resistance properties of the rubber mixture of the invention. In particularly advantageous embodiments of the invention, SSBR is used in combination with at least one further rubber to achieve an optimal and balanced profile of properties.

It is preferable when the rubber mixture contains at least one filler, preferably in amounts of 30 to 500 phr, more preferably 50 to 400 phr, in turn preferably 80 to 300 phr.

In advantageous embodiments of the invention, the filler is a reinforcing filler which is preferably selected from the group consisting of carbon blacks and silicon dioxide.

Suitable carbon blacks include any carbon black types known to those skilled in the art. It is preferable when the carbon black is selected from industrial carbon blacks and pyrolysis carbon blacks, wherein industrial carbon blacks are more preferred.

It is preferable when the carbon black has an iodine number according to ASTM D 1510, also known as the iodine adsorption number, between 30 and 250 g/kg, preferably 30 to 180 g/kg, more preferably 40 to 180 g/kg, and even more preferably 40 to 130 g/kg, and a DBP number according to ASTM D 2414 of 30 to 200 ml/100 g, preferably 70 to 200 ml/100 g, more preferably 90 to 200 ml/100 g.

The DBP number in accordance with ASTM D 2414 determines the specific absorption volume of a carbon black or a light-colored filler by means of dibutyl phthalate.

The use of such a type of carbon black in the rubber mixture, in particular for vehicle tires, ensures the best possible compromise between abrasion resistance and heat buildup, which in turn influences the ecologically relevant rolling resistance.

A particularly suitable and preferred carbon black is one having an iodine adsorption number between 80 and 110 g/kg and a DBP number of 100 to 130 ml/100g, such as in particular carbon black of the N339 type.

The silicon dioxide is preferably amorphous silicon dioxide, for example precipitated silica, which is also referred to as precipitated silicon dioxide. However, it is alternatively also possible to use fumed silicon dioxide for example.

However, particular preference is given to using a finely divided, precipitated silica which has a nitrogen surface area (BET surface area) (according to DIN ISO 9277 and DIN 66132) of 35 to 400 m²/g, preferably of 35 to 350 m²/g, more preferably of 85 to 320 m²/g and even more preferably of 120 to 235 m²/g and a CTAB surface area (according to ASTM D 3765) of 30 to 400 m²/g, preferably of 30 to 330 m²/g, more preferably of 80 to 300 m²/g and even more preferably of 115 to 200 m²/g. Such silicas lead, for example in rubber mixtures for tire treads, to particularly good physical properties of the vulcanizates. Advantages in mixture processing by way of a reduction in mixing time can also arise here while retaining the same product properties, leading to improved productivity. Silicas used may thus, for example, be either those of the Ultrasil® VN3 type (trade name) from Evonik or highly dispersible silicas known as HD silicas (e.g. Zeosil® 1165 MP from Solvay).

In particularly advantageous embodiments of the invention, the rubber mixture contains at least one silica as filler, preferably in amounts of 30 to 500 phr, more preferably 50 to 400 phr, in turn preferably 80 to 300 phr.

In these quantities, silica is especially present as the sole or primary filler (more than 50% by weight based on total filler amount).

In further advantageous embodiments of the invention, the rubber mixture contains at least one silica as further filler, preferably in amounts of 5 to 100 phr, more preferably 5 to 80 phr, in turn preferably 10 to 60 phr.

In these quantities, silica is especially present as a further filler in addition to another primary filler, such as in particular a carbon black.

The terms “silicic acid” and “silica” are used synonymously in the context of the present invention.

In particularly advantageous embodiments of the invention the rubber mixture according to the invention contains 0.1 to 60 phr, preferably 3 to 40 phr, particularly preferably 5 to 30 phr, very particularly preferably 5 to 15 phr, of at least one carbon black. In these quantities, carbon black is especially present as a further filler in addition to a primary filler, such as in particular silica.

In a further advantageous embodiment of the invention the rubber mixture according to the invention contains 30 to 300 phr, preferably 30 to 200 phr, particularly preferably 40 to 100 phr, of at least one carbon black. In these quantities carbon black is present as the sole or primary filler and is therefore optionally present in combination with silica in the abovementioned smaller amounts.

In a particularly advantageous embodiment of the invention, the rubber mixture contains 5 to 60 phr, more preferably 5 to 40 phr, of at least one carbon black and 50 to 300 phr, preferably 80 to 200 phr, of at least one silica.

The rubber mixture may additionally contain further fillers which are reinforcing or non-reinforcing.

The further (non-reinforcing) fillers in the context of the present invention include aluminosilicates, kaolin, chalk, starch, magnesium oxide, titanium dioxide or rubber gels, and fibers (for example aramid fibers, glass fibers, carbon fibers, cellulose fibers).

Further, optionally reinforcing fillers are for example carbon nanotubes ((CNTs), including discrete CNTs, hollow carbon fibers (HCF) and modified CNTs comprising one or more functional groups such as hydroxy, carboxy and carbonyl groups), graphite and graphene and what is known as “carbon-silica dual-phase filler”.

In the context of the present invention zinc oxide is not included among the fillers.

The rubber mixture may further contain customary additives in customary parts by weight which are added preferably in at least one primary mixing stage during the production of said mixture. These additives include
a) aging stabilizers known in the prior art,
   such as for example p-phenylenediamine, such as N-phenyl-N′-(1,3-dimethylbutyl)-p-phenylenediamine (6PPD), N,N′-diphenyl-p-phenylenediamine (DPPD), N-(1-phenylethyl)-N′-phenyl-p-phenylenediamine (SPPD), N,N′-ditolyl-p-phenylenediamine (DTPD), N-(1,4-dimethylpentyl)-N′-phenyl-p-phenylenediamine (7PPD), N-isopropyl-N′-phenyl-p-phenylenediamine (IPPD),
   or dihydroquinolines, such as 2,2,4-trimethyl-1,2-dihydroquinoline (TMQ),
b) activators, for example zinc oxide and fatty acids (e.g. stearic acid) and/or other activators, such as zinc complexes, for example zinc ethylhexanoate,
c) activators and/or agents for binding fillers, in particular carbon black or silica, for example S-(3-aminopropyl) thiosulfuric acid and/or metal salts thereof (bonding of carbon black) and silane coupling agents (binding to silicon dioxide, in particular silica),
d) antiozonant waxes,
e) resins, especially tackifying resins,
f) masticating aids, for example 2,2′-dibenzamidodiphenyl disulfide (DBD), and
g) processing aids, such as in particular fatty acid esters and metal soaps, for example zinc soaps and/or calcium soaps,
h) plasticizers, such as in particular aromatic, naphthenic or paraffinic mineral oil plasticizers, for example MES (mild extraction solvate) or RAE (residual aromatic extract) or TDAE (treated distillate aromatic extract), or rubber-to-liquid oils (RTL) or biomass-to-liquid oils (BTL) preferably having a content of polycyclic aromatics of less than 3% by weight according to method IP 346 or triglycerides, for example rapeseed oil or factices or hydrocarbon resins or liquid polymers having a mean molecular weight (determination by GPC=gel permeation chromatography, in accordance with BS ISO 11344:2004) between 500 and 20 000 g/mol.

When using mineral oil this is preferably selected from the group consisting of DAE (distillate aromatic extracts), RAE (residual aromatic extract), TDAE (treated distillate aromatic extracts), MES (mild extracted solvents) and naphthenic oils.

In particularly advantageous embodiments the rubber mixture according to the invention contains no aging stabilizers from the group of p-phenylenediamines, in particular those listed above under a), in addition to the inventive compound of formula I), for example of formula III). In a particularly preferred embodiment the rubber mixture according to the invention especially contains 0 to 0.1 phr, in particular 0 phr, of further aging stabilizers based on p-phenylenediamines and selected from the group containing, preferably consisting of, N-phenyl-N′-(1,3-dimethylbutyl)-p-phenylenediamine (6PPD), N-(1-phenylethyl)-N′-phenyl-p-phenylenediamine (SPPD), N,N′-diphenyl-p-phenylenediamine (DPPD), N,N′-ditolyl-p-phenylenediamine (DTPD), N-isopropyl-N′-phenyl-p-phenylenediamine (IPPD), N-(1,4-dimethylpentyl)-N′-phenyl-p-phenylenediamine (7PPD).

The preferably very small amounts of 0 to 0.1 phr, particularly preferably 0 phr, of p-phenylenediamines, and the compound of formula I), for example of formula III), present according to the invention makes it possible to achieve a comparable protective effect at lower toxicity. The inventive compound of formula I), for example of formula III), replaces the recited p-phenylenediamines known in the prior art.

In further advantageous embodiments of the invention, at least one further representative of the recited p-phenylenediamine aging stabilizers is present, and so the compound of the invention only partially replaces the p-phenylenediamines known in the prior art. This also achieves the advantage of the invention, just not to an optimal extent.

In advantageous embodiments aging stabilizers based on dihydroquinoline, such as TMQ are present in the rubber mixture in addition to the inventive compound of formula I). The amount of dihydroquinolines present, such as especially TMQ is preferably 0.1 to 3, in particular 0.5 to 1.5, phr.

Antiozonant waxes (group d above) are considered separately and in preferred embodiments of the invention are present in the rubber mixture irrespective of whether additional aging stabilizers a) are present.

The silane coupling agents may be any of the types known to those skilled in the art.

Furthermore, one or more different silane coupling agents may be used in combination with one another. The rubber mixture may thus contain a mixture of different silanes.

The silane coupling agents react with the surface silanol groups of the silicon dioxide, in particular of the silica, or other polar groups during the mixing of the rubber/the rubber mixture (in situ) or in the context of a pretreatment (premodification) even before addition of the filler to the rubber.

Coupling agents known from the prior art are bifunctional organosilanes having at least one alkoxy, cycloalkoxy or phenoxy group as a leaving group on the silicon atom and having as another functionality a group which, possibly after cleavage, can enter into a chemical reaction with the double bonds of the polymer. The latter group may for example comprise the following chemical groups:
−SCN, −SH, −NH₂or −Sₓ₋ (with x=2 to 8).

Employable silane coupling agents thus include for example 3-mercaptopropyltriethoxysilane, 3-thiocyanatopropyltrimethoxysilane or 3,3′-bis(triethoxysilylpropyl) polysulfides having 2 to 8 sulfur atoms, for example 3,3′-bis(triethoxysilylpropyl) tetrasulfide (TESPT), the corresponding disulfide (TESPD), or else mixtures of the sulfides having 1 to 8 sulfur atoms with different contents of the various sulfides. TESPT may for example also be added as a mixture with industrial carbon black (trade name X50S® from Evonik).

Blocked mercaptosilanes as known for example from WO 99/09036 may also be used as a silane coupling agent. It is also possible to use silanes as described in WO 2008/083241 A1, WO 2008/083242 A1, WO 2008/083243 A1 and WO 2008/083244 A1. Usable silanes include, for example, those sold by Momentive, USA in a number of variants under the NXT name, such as especially 3-octanoylthio-1-propyltriethoxysilane, or those sold by Evonik Industries under the VP Si 363® name.

The total proportion of further additives is preferably 3 to 150 phr, more preferably 3 to 100 phr and most preferably 5 to 80 phr.

Zinc oxide (ZnO) may be included in the total proportion of further additives in the abovementioned amounts.

This may be any type of zinc oxide known to those skilled in the art, for example ZnO granules or powder. The zinc oxide conventionally used generally has a BET surface area of less than 10 m²/g. However, it is also possible to use a zinc oxide having a BET surface area of 10 to 100 m²/g, for example “nano zinc oxides”.

The rubber mixture of the invention is preferably used in vulcanized form, in particular in vehicle tires or other vulcanized industrial rubber articles.

The terms “vulcanized” and “crosslinked” are used synonymously in the context of the present invention.

The vulcanization of the rubber mixture of the invention is preferably conducted in the presence of sulfur and/or sulfur donors with the aid of vulcanization accelerators, it being possible for some vulcanization accelerators to act simultaneously as sulfur donors. The accelerator is selected from the group consisting of thiazole accelerators, mercapto accelerators, sulfenamide accelerators, thiocarbamate accelerators, thiuram accelerators, thiophosphate accelerators, thiourea accelerators, xanthogenate accelerators and guanidine accelerators.

It is preferable to use a sulfenamide accelerator selected from the group consisting of N-cyclohexyl-2-benzothiazolesulfenamide (CBS), N,N-dicyclohexylbenzothiazole-2-sulfenamide (DCBS), benzothiazyl-2-sulfenomorpholide (MBS), N-tert-butyl-2-benzothiazylsulfenamide (TBBS) and guanidine accelerators such as diphenylguanidine (DPG).

The sulfur donor substance employed may be selected from any of the sulfur donor substances known to those skilled in the art.

Vulcanization retarders may also be present in the rubber mixture.

The production of the rubber mixture of the invention is preferably otherwise effected by the process customary in the rubber industry in which a primary mixture comprising all constituents except the vulcanization system (for example sulfur and vulcanization-influencing substances) is first produced in one or more mixing stages. The finished mixture is produced by adding the vulcanization system in a final mixing stage.

The finished mixture is for example processed further and brought into the appropriate shape by means of an extrusion operation or calendering.

The rubber mixture of the invention is particularly suitable for use in vehicle tires, especially pneumatic vehicle tires. Use in all tire components is conceivable in principle, in particular in an outer component, in particular and preferably in the flange profile, tread and/or sidewall. In the case of a tread with cap/base construction, the rubber mixture of the invention is preferably used at least in the cap.

For use in vehicle tires, the mixture as a finished mixture prior to vulcanization is brought into the corresponding shape, preferably of an outer component, and during production of the green vehicle tire is applied in the known manner.

The production of the rubber mixture of the invention for use as any other body mixture in vehicle tires is effected as described above. The difference lies in the shaping after the extrusion operation/the calendering of the mixture. The shapes thus obtained of the as-yet unvulcanized rubber mixture for one or more different body mixtures then serve for the construction of a green tire.

“Body mixture” refers here to the rubber mixtures for the inner components of a tire, such as essentially squeegee, inner liner (inner layer), core profile, belt, shoulder, belt profile, carcass, bead reinforcement, bead profile, flange profile and bandage.

The as-yet unvulcanized green tire is subsequently vulcanized.

For use of the rubber mixture of the invention in drive belts and other belts, especially in conveyor belts, the extruded, as-yet unvulcanized mixture is brought into the appropriate shape and often provided at the same time or subsequently with strength members, for example synthetic fibers or steel cords. This usually affords a multi-ply construction consisting of one and/or more plies of rubber mixture, one and/or more plies of identical and/or different strength members and one and/or more further plies of the same and/or another rubber mixture.

The present invention further provides a vehicle tire which comprises the rubber mixture according to the invention containing the compound according to the invention in at least one component.

The vulcanized vehicle tire, in at least one component, comprises a vulcanizate of at least one rubber mixture of the invention. It is known to those skilled in the art that most substances, for example the rubbers present, are present or may be present in chemically modified form either already after mixing or only after vulcanization.

In the context of the present invention, “vehicle tires” mean pneumatic vehicle tires and all-rubber tires, including tires for industrial and construction site vehicles, truck, car and two-wheeler tires.

It is preferable when the vehicle tire of the invention comprises the rubber mixture of the invention in at least one outer component, wherein the outer component is preferably a tread, a sidewall and/or a flange profile.

The vehicle tire according to the invention may accordingly contain the rubber mixture of the invention containing the inventive compound of formula I) in two or more components, optionally with an adjusted composition.

The invention shall now be more particularly elucidated with reference to exemplary embodiments.

The compound of formula III) as an exemplary embodiment of the compound of formula I) was produced in the following manner as shown in schemes S1a) and S2):

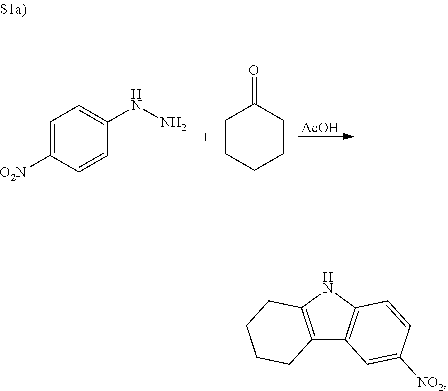 [SMILES: "AcOH" ; NNc1ccc([N+](=O)[O-])cc1 ; O=C1CCCCC1 ; O=[N+]([O-])c1ccc2[nH]c3c(c2c1)CCCC3]

wherein AcOH stands for acetic acid and the synthesis was carried out according to scheme S1a) according to B.A. Dalvi, Tet. Lett., 2018, 59, 2145-2149.

### Synthesis of 6-(1,3-dimethylbutylamino)-2,3,4,9-tetrahydro-1H-carbazole (Compound of Formula III):

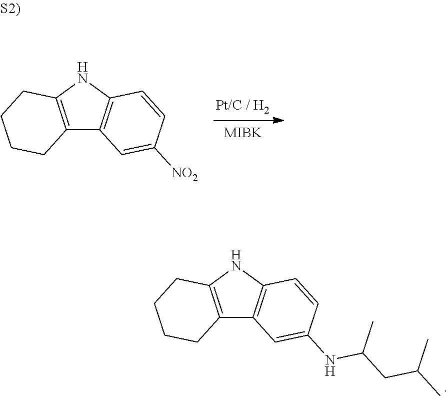 [SMILES: O=[N+]([O-])c1ccc2[nH]c3c(c2c1)CCCC3 ; CC(C)CC(C)Nc1ccc2[nH]c3c(c2c1)CCCC3]

0.30 g (1.39 mmol, 1 equivalent (eq.)) of 6-nitro-2,3,4,9-tetrahydro-1H-carbazole, 0.12 g of platinum on carbon (5%) (0.4 g on 4.67 mmol of substrate) and 20.0 ml of methyl isobutyl ketone were weighed into a stainless steel autoclave fitted with a Teflon inliner. The reaction mixture was subsequently subjected to hydrogen at a pressure of 20 bar and stirred for 10 hours at 60° C. After the reaction had ended, the excess hydrogen was released and the suspension was filtered through Celite® and washed with ethanol. The filtrate was concentrated to dryness and dried under vacuum. Grayish solid; yield 0.37 g (98% of theory).

¹H-NMR (nuclear magnetic resonance) (500 MHz, DMSO-d6)δ=10.07 (s, 1H), 6.95 (d, J=8.5 Hz, 1H), 6.45 (d,J=2.1 Hz, 1H), 6.39 (dd, J=8.5, 2.1 Hz, 1H), 4.36 (s, 1H), 3.41 (q,J=6.4 Hz, 1H), 2.63 (t,J=5.9 Hz, 2H), 1.84−1.69 (m, 7H), 1.46 (dt,J=13.8, 7.0 Hz, 1H), 1.18 (dt,J=13.5, 6.8 Hz, 1H), 1.07 (d,J=6.1 Hz, 3H), 0.92 (d,J=6.6 Hz, 3H), 0.90 (d,J=6.6 Hz, 3H).

¹³C-NMR (126 MHz, DMSO-d6)δ=141.7, 134.5, 129.5, 128.6, 111.3, 110.5, 107.4, 99.8, 47.2, 46.7, 26.8, 25.1, 23.6, 23.5, 23.4, 23.4, 23.0, 21.3, 21.3.

ESI-MS (electrospray ionization mass spectrometry) [M+H]⁺=271.

### Measurement of Oxidation Induction Time (OIT)

The compound of formula III) was investigated under laboratory conditions for its potential protective effect as an aging stabilizer by measurement of the oxidation induction time.

To this end the compounds of formula III) and 6PPD in each case together with a polymer (liquid synthetic polyisoprene (IR), LIR-50, Kuraray, weight average molecular weight distribution M_{w}=54 000 g/mol, glass transition temperature T_{g}=−63° C.) were heated at constant temperature (180° C.) until onset of oxidation (start temperature 35° C., heating to 170° C. at a heating rate of 20 K/min (kelvin per minute), heating to 180° C. at a heating rate of 1 K/min; purge gas: nitrogen (N₂), volume flow 50 ml/min).

The specimen was kept under isothermal conditions at 180° C. under an N2 atmosphere for 5 minutes and the atmosphere was then switched to an O2 atmosphere (volume flow 50 ml/min).

Oxidation was determined via a peak using DSC (differential scanning calorimetry).

The time in minutes to oxidation was measured.

The results compared to the known aging stabilizer 6PPD are summarized in table 1.

TABLE 1

| | Time [min] |
|---|---|
| Substance | at 180° C. |
| 6PPD | 116 ± 10 |
| Formula III) | 119 ± 10 |

Taking into account the measurement accuracy of ± (plus/minus) 10 minutes it is apparent that the compound of formula III) achieves a comparable antioxidant effect to 6PPD at 180° C.

The inventive compound of formula III) as a representative of the inventive compound of formula I) is thus more environmentally friendly and less harmful to health than 6PPD/further representatives of the substance class as mentioned above and is also a comparable aging stabilizer.

At the same time the inventive compound of formula III) as a representative of the inventive compound of formula I) exhibits a very good solubility in rubber mixtures. This prevents blooming, which in turn entails an improved protective effect.

The compound of formula I) thus makes it possible to achieve a comparable or, in terms of the reduced blooming behavior, even an improved protective effect for the recited possible applications.

For use in a rubber mixture for vehicle tires the inventive compound of formula I), for example of formula III), is added in one of the mixing stages during production of the rubber mixture in a manner known to those skilled in the art for example instead of the aging stabilizers known in the prior art, such as 6PPD, 7PPD or IPPD etc.

To this end the compound of formula III) is by way of example incorporated in various amounts as shown in table 2. The resultant inventive examples are identified as E1 and E2.

As a comparison, rubber mixtures containing 6PPD instead of the compound of formula III) are employed as aging stabilizers at otherwise identical composition, with substitution in each case on a mole-per-mole basis between V1 and E1 and V2 and E2. The amounts in table 2 are reported in units of phr. A reference (Ref.) without aging stabilizer is also reported.

In all mixtures the sum of the amounts of aging stabilizer (6PPD or formula III) and plasticizer oil MES is 10 phr.

TABLE 2

| Constituent | V1 | E1 | V2 | E2 | Ref. |
|---|---|---|---|---|---|
| NR | 100 | 100 | 100 | 100 | 100 |
| N 339 | 50 | 50 | 50 | 50 | 50 |
| carbon | | | | | |
| black | | | | | |
| MES | 8.0 | 8.0 | 5.0 | 5.0 | 10 |
| 6PPD | 2.0 | — | 5.0 | — | — |
| Compound | — | 2.0 | — | 5.0 | — |
| of formula | | | | | |
| III) | | | | | |
| ZnO | 3 | 3 | 3 | 3 | 3 |
| Stearic acid | 2 | 2 | 2 | 2 | 2 |
| TBBS | 1.2 | 1.2 | 1.2 | 1.2 | 1.2 |
| Sulfur | 1.2 | 1.2 | 1.2 | 1.2 | 1.2 |

The inventive examples show a comparable or even improved aging stabilization effect compared to rubber mixtures comprising 6PPD.

1. A compound of formula I): 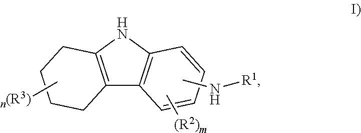
wherein R¹ is selected from the group consisting of xi) aromatic radicals, wherein the aromatic radicals optionally bear substituents selected from the group consisting of halogen radicals, cyano radicals, ester radicals, ketone radicals, ether radicals and thioether radicals,
and xii) linear, branched and cyclic aliphatic C₃-to C₁₂-radicals,
wherein the radicals R² and R³ may independently of one another be identical or different and are selected from the group consisting of linear, branched and cyclic, saturated and unsaturated, aliphatic C₁-to C₁₂-radicals optionally bearing one or more halogen substituents, aryl radicals optionally bearing one or more halogen substituents, and halogen radicals, wherein fluorine, bromine and chlorine are preferred, cyano radicals, ester radicals, ketone radicals, ether radicals and thioether radicals
and wherein m assumes the value 0 or 1 or 2 or 3
and wherein n assumes the value 0 or is an integer from 1 to 8, preferably 1 to 4.

2. The compound as claimed in claim 1, wherein it has the structure of formula II): 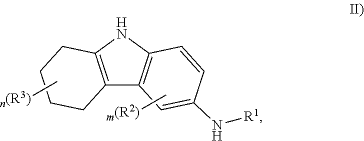
wherein R¹, R², R³ and m and n are as defined in claim 1.

3. The compound as claimed in claim 1, wherein m is zero (0) and/or n is zero (0).

4. The compound as claimed in claim 1, wherein R¹ is bonded to the nitrogen atom (N) via a tertiary carbon atom.

5. The compound as claimed in claim 1, wherein R¹ is a branched and/or cyclic alkyl radical having 3 to 12 carbon atoms, preferably 3 to 8 carbon atoms.

6. The compound as claimed in claim 1, wherein R¹ is selected from 1,3-dimethylbutyl and cyclohexyl radicals, wherein R¹ is preferably a 1,3-dimethylbutyl radical.

7. The compound as claimed in claim 1, wherein it has the structure of formula III):

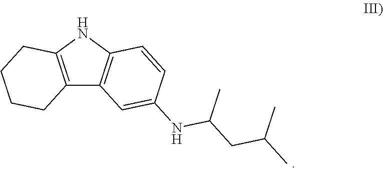 [SMILES: CC(C)CC(C)Nc1ccc2[nH]c3c(c2c1)CCCC3]

8. A rubber mixture containing the compound as claimed in claim 1,
wherein the rubber mixture preferably contains at least one diene rubber which is particularly preferably selected from the group consisting of natural polyisoprene (NR), synthetic polyisoprene (IR), butadiene rubber (BR), solution-polymerized styrene-butadiene rubber (SSBR), emulsion-polymerized styrene-butadiene rubber (ESBR), butyl rubber (IIR) and halobutyl rubber.

9. A vehicle tire which comprises the rubber mixture as claimed in claim 8 in at least one component, preferably in at least one external component, wherein the external component is preferably a tread, a sidewall and/or a flange profile.

10. A method of using the compound as claimed in claim 1 comprising: providing the compound and using the compound as an aging stabilizer and/or antiozonant especially in vehicle tires and/or other industrial rubber articles, such as especially an air spring, a bellows, a conveyor belt, a strap, a drive belt, a hose, a rubber band, a profile, a seal, a membrane, tactile sensors for medical applications or robotics applications or a shoe sole or parts thereof and/or oils and/or lubricants.

11. A method of using the compound as claimed in claim 1 comprising: providing the compound and using the compound for producing a rubber article, in particular an air spring, a bellows, a conveyor belt, a strap, a drive belt, a hose, a rubber band, a profile, a seal, a membrane, tactile sensors for medical applications or robotics applications or a shoe sole or parts thereof.

12. A process for producing the compound of formula I) which comprises at least the following process steps:
i) producing or providing the substance of formula B1): 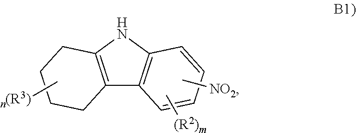
ii) reacting the compound of formula B1) with hydrogen and a ketone or aldehyde, preferably ketone, to afford the compound of formula I): 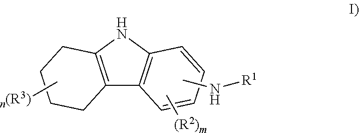
wherein R¹ is selected from the group consisting of xi) aromatic radicals, wherein the aromatic radicals optionally bear substituents selected from the group consisting of halogen radicals, cyano radicals, ester radicals, ketone radicals, ether radicals and thioether radicals,
and xii) linear, branched and cyclic aliphatic C₃-to C₁₂-radicals,
wherein the radicals R² and R³ may independently of one another be identical or different and are selected from the group consisting of linear, branched and cyclic, saturated and unsaturated, aliphatic C₁-to C₁₂-radicals optionally bearing one or more halogen substituents, aryl radicals optionally bearing one or more halogen substituents, and halogen radicals, wherein fluorine, bromine and chlorine are preferred, cyano radicals, ester radicals,
ketone radicals, ether radicals and thioether radicals
and wherein m assumes the value 0 or 1 or 2 or 3
and wherein n assumes the value 0 or is an integer from 1 to 8, preferably 1 to 4.

13. The process as claimed in claim 12, wherein the reaction in step ii) is carried out with hydrogen using a hydrogenation catalyst and/or at a temperature of 50° C. to 130° C., preferably 50° C. to 100° C., particularly preferably 50° C. to 80° C., and/or the reaction mixture is subjected to hydrogen at a pressure of 10 to 70 bar, particularly preferably 10 to 30 bar, in particular 20 bar for example, and the reaction is carried out in an autoclave or in another pressure reactor.

14. The process as claimed in claim 12, wherein the compound of formula I) has the structure of formula II): 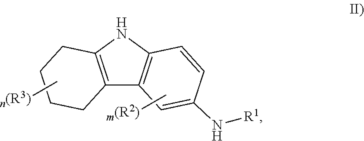
and wherein the substance of formula B1) has the structure of formula B1a): [chemical structure]
